(19) United States
(12) Patent Application Publication
(10) Pub. No.: US 2026/0171233 A1
(43) Pub. Date: Jun. 18, 2026

(54) RECOVERING AN IMPLANTABLE NEUROSTIMULATOR FROM UNEXPECTED OPERATIONAL STATES

(71) Applicant: MEDTRONIC, INC., Minneapolis, MN (US)

(72) Inventors: Emily G. Klotsche, St. Paul, MN (US); Saketh Karumuri, Maple Grove, MN (US); Kunal J. Paralikar, Roseville, MN (US); Naga Deepthi Nannapaneni, Leander, TX (US); Abdulrahman W. Aref, Stillwater, MN (US)

(21) Appl. No.: 19/417,464

(22) Filed: Dec. 12, 2025

Related U.S. Application Data
(60) Provisional application No. 63/733,332, filed on Dec. 12, 2024.

(51) Int. Cl. G16H 40/40

(52) U.S. Cl. CPC G16H 40/40

(57) ABSTRACT

Techniques of the present disclosure generally relate to recovering a medical device from unexpected operational states, and more particularly for recovering an implantable medical device (IMD), such as a neurostimulator, from unexpected operational states while retaining service life and diagnostic data information. This advantageously prevents or reduces costly and inconvenient device explant or field corrective actions. A recovery operation may include generating an authentication code corresponding to the IMD, transmitting the authentication code to an external source, receiving an authentication key from the external source, reading the authentication key to validate the authentication code, resetting one or more device operation settings while retaining service life and diagnostic data information in a device memory of the IMD, and writing, from the device memory, the service life information and a default parameter corresponding to the one or more reset device operation settings to an instrument memory of the IMD.

### CROSS-REFERENCE TO RELATED APPLICATIONS

This application claims the benefit of and priority to U.S. Provisional Patent Application Ser. No. 63/733,332 filed Dec. 12, 2024, the entire disclosure of which is incorporated by reference herein.

### FIELD

The present technology generally relates to systems, devices, and methods for recovering a medical device from unexpected operational states. More particularly, the present technology is related to systems, devices, and methods for recovering an implantable medical device, such as a neurostimulator, from unexpected operational states while retaining existing service life and existing diagnostic data information.

### BACKGROUND

Implantable neurostimulators, which are a class of implantable medical devices (IMDs), are used in various medical procedures and therapies, including those related to pelvic health such as sacral and tibial neuromodulation, to deliver electrical stimulation to targeted nerves. Targeted electrical stimulation of nerves can be used for the management and treatment of various neurological disorders and chronic pain conditions. For example, chronic pain and movement disorders such as Parkinson's disease, epilepsy, certain psychiatric conditions, and pelvic maladies have been areas of significant focus for neurostimulation therapies. Traditional treatment modalities for these conditions may involve pharmacological interventions which can have limited efficacy and are frequently associated with adverse side effects. As a result, there is a growing demand for alternative therapies that offer more precise, targeted, and effective management of symptoms.

Most IMDs are programmed and monitored using software such as a clinician programmer application. A clinician programmer application enables healthcare professionals to program or adjust stimulation therapy settings to provide adequate nerve stimulation and sufficient comfort during therapy. The clinician programmer application can also perform data integrity checks on the IMD, for example cyclic redundancy checks, to ensure proper device operation based on data stored within the device. Generally, the clinician programmer application has built-in mechanisms to correct certain invalid data fields should they be encountered during data integrity checks. However, these built-in mechanisms fall short in addressing all instances of invalid data fields, particularly unforeseen scenarios that currently necessitate IMD explant, field corrective actions, or unplanned software updates. The cost and inconvenience of these conventional correction procedures are substantial, impacting not only the patient who must endure the physical and emotional strain of the procedure, but also healthcare professionals who face the complexities and risks associated with performing the explant procedure or corrective action in the field.

Alternative correction procedures that do not require explant or field corrective action would provide improved outcomes and satisfaction for both patients and healthcare professionals. Patients would benefit from reduced physical and emotional stress by avoiding invasive procedures and lengthy recovery times. This would lead to a quicker return to normal activities and a better overall quality of life. For healthcare professionals, alternative correction procedures would reduce the risks inherent in surgical interventions, streamline workflows, and allow for more efficient allocation of resources and time. This would not only enhance the quality of care provided to patients, but also increase healthcare professional satisfaction by eliminating the complexities and uncertainties involved in conventional corrective procedures.

In light of this, the inventors of the present disclosure have developed techniques to address unforeseen invalid data scenarios without requiring IMD explant or field corrective actions. The present disclosure describes and illustrates these techniques along with various advantages they provide over conventional techniques.

### SUMMARY

The techniques of the present disclosure generally relate to recovering a medical device from unexpected operational states, and more particularly for recovering an IMD, such as a neurostimulator, from unexpected operational states while retaining existing service life and existing diagnostic data information. Techniques of the present disclosure advantageously prevent or reduce device explant or field corrective actions.

Non-limiting examples of the present disclosure provide a method for recovering an implantable medical device (IMD) from unexpected operational states. The method can include generating, from a programmer application, an authentication code corresponding to the IMD; transmitting, via the programmer application, the authentication code to an external source; receiving, via the programmer application, an authentication key from the external source; reading, via the programmer application, the authentication key to validate the authentication code; resetting, upon validating the authentication code, one or more device operation settings stored in a device memory of the IMD, wherein existing service life and existing diagnostic data of the IMD are maintained in the device memory during the reset; and writing, from the device memory, the existing service life and at least one default parameter corresponding to the one or more reset device operation settings to an instrument memory of the IMD.

Non-limiting examples of the present disclosure provide a system including a programmer application and an implantable medical device (IMD). The IMD can include a device memory and an instrument memory. The programmer application can be configured to generate an authentication code corresponding to the IMD; transmit the authentication code to an external source; receive an authentication key from the external source; read the authentication key to validate the authentication code; reset, upon validating the authentication code, one or more device operation settings stored in the device memory, wherein existing service life and existing diagnostic data of the IMD are maintained in the device memory during the reset; and write, from the device memory, the existing service life and at least one default parameter corresponding to the one or more reset device operation settings to the instrument memory.

Non-limiting examples of the present disclosure provide an implantable medical device (IMD) including a device memory having one or more device operation settings and an instrument memory. The device memory can be configured to reset the one or more device operation settings upon being instructed to. Existing service life and existing diagnostic data of the IMD can be maintained in the device memory during the reset. The instrument memory can be configured to receive the existing service life and at least one default parameter corresponding to the one or more reset device operation settings from the device memory.

Non-limiting examples of the present disclosure provide a computer-readable medium storing a set of instructions which, when executed by a processor, cause the processor to: generate an authentication code corresponding to IMD; transmit the authentication code to an external source; receive an authentication key from the external source; read the authentication key to validate the authentication code; reset, upon validating the authentication code, one or more device operation settings stored in a device memory of the IMD, wherein existing service life and existing diagnostic data of the IMD are maintained in the device memory during the reset; and write, from the device memory, the existing service life and at least one default parameter corresponding to the one or more reset device operation settings to an instrument memory of the IMD.

Advantages of the present disclosure include: preventing or reducing IMD explants, field corrective actions, and software updates due to unexpected operational states or other challenges encountered by the device; providing the ability to clear and reprogram all device operation settings, thereby giving the appearance of a factory reset device, while retaining the existing service life and diagnostic data of the device; giving medical device manufacturers and healthcare providers the ability to better serve patients by incorporating a mechanism where the patients can start an intentional troubleshooting session if unexpected operational states or other device challenges are encountered; providing a secure, encrypted validation method requiring medical device manufacturer or healthcare professional intervention before starting an operation for recovering an IMD from an unexpected operational state (to ensure that device clearing and reprograming is required); providing the option to reprogram a reset IMD with default device parameters or previously used therapy parameters; enabling manual, semi-autonomous, or fully autonomous intervention and recovery of an IMD from an unexpected operational state; providing methods for recovering from unexpected operational states that are compatible with a variety of IMDs including neurostimulators; and providing less costly and more convenient solutions to correct unexpected operational states and other device challenges compared to conventional techniques (e.g., explants, field corrective actions, dynamic device reset and reprograming operations, factory-like reset and reprograming).

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 illustrates a first method for recovering an implantable medical device from unexpected operational states, according to examples of the present disclosure;

FIG. 2 illustrates a second method for recovering an implantable medical device from unexpected operational states, according to examples of the present disclosure;

FIG. 3 illustrates a third method for recovering an implantable medical device from unexpected operational states, according to examples of the present disclosure;

FIG. 4 illustrates a first flowchart for recovering an implantable medical device from unexpected operational states, according to examples of the present disclosure;

FIG. 5 illustrates a block diagram of a system configured to recover an implantable medical device from unexpected operational states, according to examples of the present disclosure;

FIG. 6 illustrates an implantable medical device configured to recover from unexpected operational states, according to examples of the present disclosure;

FIGS. 7A-D illustrate graphical user interface screens of a programmer application configured for use with an implantable medical device to recover the implantable medical device from unexpected operational states, according to examples of the present disclosure;

FIGS. 8A and 8B illustrate graphical user interface screens of an authentication key generator program for use with a programmer application to recover an implantable medical device from unexpected operational states, according to examples of the present disclosure; and

FIG. 9 illustrates a second flowchart for recovering an implantable medical device from unexpected operational states, according to examples of the present disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

Examples of the present disclosure include systems, devices, and methods for recovering an implantable medical device (IMD) from unexpected operational states without requiring IMD explant, field corrective actions, or unplanned software updates to the IMD or a programmer application couplable to the IMD.

Generally, whenever an IMD is interrogated or functionally analyzed after implantation, a programmer application conducts periodic data validation checks as well as other operation checks (e.g., cyclic redundancy checks (CRCs) to ensure the IMD is operating as expected). This can comprise error-detecting code configured to detect accidental or intentional changes to data. The data validation checks are also intended to verify the integrity of data stored within the IMD, for example existing diagnostic data corresponding to a patient carrying the IMD. The programmer application may be available to the patient carrying the IMD, to a healthcare professional advising the patient, to a manufacturer of the IMD, or to all three entities simultaneously.

The programmer application can comprise built-in data correction mechanisms to remedy certain foreseen invalid data fields known to the application (e.g., invalid data fields previously encountered by the IMD or by other patients carrying the same IMD). However, the built-in data correction mechanisms cannot account for all invalid data fields including those that are unforeseen because the IMD is operating in an unexpected state. Unexpected states can include any invalid or corrupted data field, or unforeseen operating state, without a built-in correction mechanism. Examples of unexpected states include: random bursts of cosmic radiation which can corrupt memory units of the IMD or the programmer application, thereby rendering the device or application unrecoverable using built-in mechanisms; rare and unlikely intruder scenarios such as a third-party inputting random, unexpected device settings based on intercepted communications or instructions to the IMD or the programmer application; a compromised programmer application writing invalid device settings; a future design gap not currently anticipated; and programmer application defects resulting in interrogation loops that prevent therapy adjustment. Currently, when certain IMDs encounter unexpected invalid data scenarios, the IMD or the programmer application may become unusable or behave unexpectedly until corrective measures are performed. Typical corrective measures include IMD explant and repair, field corrective actions, and unplanned software updates.

The present disclosure provides solutions to unexpected invalid data or operational state scenarios that eliminate or reduce the need for typical corrective measures. In particular, solutions of the present disclosure enable an IMD to be reset and reprogramed while retaining existing service life and existing diagnostic data information. This occurs using the existing programmer application in conjunction with an information exchange between a user such as a patient or healthcare professional and a technical services unit of a medical device manufacturer, for example. The programmer application may comprise a built-in “clear programming data” (CPD) feature configured to reset one or more IMD operation settings (e.g., all settings, except for existing service life and existing diagnostic data, which undergo periodic validation checks or CRCs) and to reprogram the IMD with default parameters corresponding to the settings. Thus, recovery of an IMD from an unexpected operational state while retaining certain device information is enabled using techniques of the present disclosure.

An IMD recovery operation may be performed according to the following process. Upon observing an unexpected operational state, the built-in CPD feature of the programmer application can be used to reset or clear one or more IMD operation settings except for existing service life and existing diagnostic data information. The one or more IMD operation settings may comprise therapy scheduler settings, stimulation settings, instrument memory information stored within the IMD, patient information, a magnetic resonance imaging (MRI) mode that enables the IMD to safely operate during an MRI scan, and other similar IMD or programmer application settings or information. Other IMD operation settings contemplated by the present disclosure include sensing settings such as amplifier settings, filter configurations, information display preferences, geographical preference settings, and other similar settings or information. These operation settings can be reset or cleared regardless of whether they are operating in expected or unexpected states. In examples, the reset or clear operation can be expanded to clear any cached application data and any report data that may not be directly present on the IMD but could be interfering with the connection or programming process of the IMD.

After resetting or clearing one or more device operation settings, the CPD feature can be used to reprogram the settings to default or existing therapy parameters (e.g., existing therapy parameters being different than default parameters). This gives the IMD the appearance of being factory reset but with existing service life and existing diagnostic data information retained. Accordingly, the IMD is reset from an unexpected operational state back to an expected operational state with core service life and diagnostic information left intact, all without requiring IMD explant or field corrective actions. Because the service life and diagnostic information are equivalent to what was found in the IMD prior to being reset, the IMD can continue operation while retaining the service life start date, expected date(s) of when device service is required, and various diagnostic data recorded during past therapy sessions with the patient. In examples, various information or data sensed by the IMD or the programmer application, and geographical preferences such as exposing certain device or application features based on a given geography and language options, may also be retained following use of the CPD feature. In examples where a firmware update is performed on the IMD, the firmware version and any data internal to the IMD firmware may also be retained following use of the CPD feature.

To activate the CPD feature, the programmer application can be configured to generate an authentication code corresponding to the implantable medical device to first validate or confirm the recovery operation. The authentication code provides a security layer to prevent unintentional or unneeded use of the CPD feature. The authentication code can be a unique, randomly generated alphanumeric code comprising six characters. The authentication code can be more or less than six characters and can be stored in an encrypted location within the programmer application. Generally, the authentication code is not maintained by the programmer application beyond its initial storage in the encrypted location for a particular recovery operation (i.e., a unique authentication code is generated for each device interrogation session and is not maintained across application sessions). The authentication code can be randomly generated by a secure random character generator, for example the Java SecureRandom Class generator. The authentication code can be generated using an application programming interface (API) such as Harmony API.

The generated authentication code can be transmitted from the programmer application to an external source configured to receive the code. The external source can be a manufacturer of the IMD, a healthcare professional, or a patient and technical service provider with access to the IMD. Upon receiving the authentication code, the external source can determine whether an IMD recovery operation is needed or whether standard troubleshooting or debugging measures can be used to resolve the reported issue. The external source can also log or record the unexpected operational state before deciding whether a recovery operation is necessary. If the external source determines that recovery using the CPD feature is necessary, the external source can send an authentication key, corresponding to the transmitted authentication code, to the programmer application. The authentication key can be generated using a decryption tool configured to unencrypt log files and to receive the authentication code generated by the programmer application (e.g., a program such as Rosetta). The authentication key generator program and the programmer application can have a shared, secret process for generating an authentication key corresponding to the authentication code.

The shared, secret process can comprise the following operations. First, a secret key can be securely retrieved from a properties file located in assets of the programmer application. The provided authentication code can be combined with the secret key to create a single concatenated string. Next, the concatenated string can be processed using a secure cryptographic hash algorithm (e.g., the SHA-256 cryptographic hash function). This can produce a fixed-length hash measured in bytes which can be subsequently encoded into a readable binary-to-text encoding scheme for further use (e.g., the Base64 string). From the encoded string, a loop can be used to extract characters to construct the expected authentication key. During this process, ambiguous characters such as “O,” “0,” “I,” and “l,” among other characters, can be deliberately skipped to ensure clarity and reduce the risk of errors. Lastly, the generated authentication key can be compared against the provided authentication code for verification purposes.

The programmer application can be configured to receive the authentication key from the external source. The programmer application can read the authentication key which can comprise comparing the authentication key received from the external source to a secret key stored within the programmer application. The secret key is generally found in source code of the programmer application, in a secure location secured by encryption technology, or in a properties file located in assets of the programmer application. Combining the secret key with the authentication code creates a single concatenated string having some connection to the authentication key (e.g., the secret key may correspond to the authentication key in a manner where the authentication key can be verified if it correlates with the secret key). In examples, comparing the authentication key to the secret key can comprise leveraging white-box cryptography to ensure the secret key remains hidden in the programmer application and cannot be extracted by users.

If the received authentication key matches the secret key (e.g., the single concatenated string is correlated to the secret key), the programmer application validates the authentication code and the IMD recovery operation can proceed (e.g., device reset and reprogramming while retaining existing service life and existing diagnostic data information). If the received authentication key does not match the secret key, the programmer application may reread or reenter the received key until a match is found. In examples, the programmer application can be configured to generate a retry limit exceeded indication if the received key is entered five times without matching the secret key. In such cases, the programmer application can be further configured to generate a new authentication code upon receiving the retry limited exceeded indication, and to transmit the new authentication code to the external source. The external source can then generate a new authentication key corresponding to the new authentication code. The authentication key reading and, if necessary, rereading processes described previously can then be repeated for the new key.

The programmer application can be configured to read existing service life information from a device memory of the IMD prior to implementing the CPD feature. After resetting or clearing all or some device operation settings, the programmer application will write default or existing therapy parameters for these settings and write the saved existing service life information into an instrument memory of the IMD. The instrument memory is generally cleared or reset prior to receiving the service life information. Generally, diagnostic data on the device memory is read-only and thus is not reset or cleared during use of the CPD feature.

The CPD feature, which enables the IMD recovery operation, is different than a factory reset because device service life and diagnostic information are maintained during the reset. Ideally, the CPD feature is not used because the IMD is designed to operate within expected states without deviation. However, rare circumstances may arise which cause the IMD to transition to an unexpected operational state, for example generating unforeseen invalid data fields that cannot be remedied with typical built-in correction mechanisms. In such rare cases, the CPD feature can be used to restore the IMD to an expected operational state without requiring costly and inconvenient IMD explant or field corrective actions. Additionally, the CPD feature can be used to provide a temporary solution to enable the IMD to continue providing patient therapy before a permanent software or firmware correction is implemented.

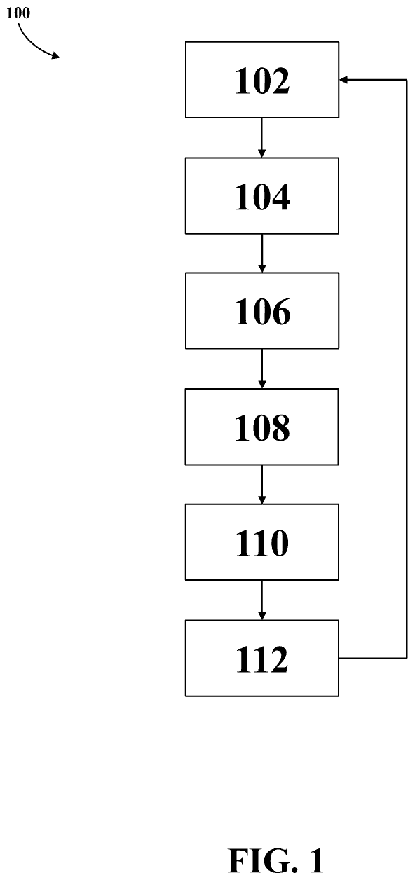

FIG. 1 illustrates a first method 100 for recovering an implantable medical device (IMD) from unexpected operational states, according to examples of the present disclosure. First method 100 may be performed using a programmer application configured to restore the IMD to an expected operational state. The programmer application may be couplable (e.g., electrically, communicatively, integrally, etc.) to the IMD. The programmer application may comprise a built-in “clear programming data” (CPD) feature configured to reset one or more IMD operation settings (e.g., all settings, except for existing service life and existing diagnostic data, which undergo periodic validation checks or cyclic redundancy checks). The programmer application may be further configured to reprogram the IMD with default parameters corresponding to the one or more reset device operation settings, along with reprogramming the retained existing service life information into the IMD.

At 102, an authentication code corresponding to the IMD may be generated using the programmer application. The authentication code may be generated manually by a user or automatically by the programmer application in response to observing an unexpected operational state of the IMD. The authentication code can be used to validate the IMD recovery operation before the operation begins. Validation is done to ensure that the CPD recovery operation is necessary to correct the observed operational state of the IMD. If the IMD recovery operation is not validated, then other built-in mechanisms of the programmer application may be used to correct certain device challenges without clearing all programming data and device operation settings. Because the IMD recovery operation is ideally never used, other, less encumbering IMD correction mechanisms should be utilized first before resorting to the recovery operation. Hence, the authentication code provides a security or protection layer to prevent unintentional or unneeded use of the CPD recovery operation when other correction mechanisms are sufficient.

The authentication code can be a unique, randomly generated alphanumeric code comprising six characters. In examples, the authentication code can be more or less than six alphanumeric characters and can be stored in an encrypted location within the programmer application. Generally, the authentication code is not maintained by the programmer application beyond its initial storage in the encrypted location for a particular CPD recovery operation. Said another way, the programmer application can be configured to generate a unique authentication code for each individual device interrogation session which is not maintained across application sessions. In examples, the authentication code can be randomly generated by a secure random character generator, for example the Java SecureRandom Class generator. The authentication code can be generated using an application programming interface (API) such as Harmony API. In general, any secure random alphanumeric character generator can be used with the programmer application to generate the authentication code.

At 104, the authentication code can be transmitted to an external source using the programmer application. The external source can be a manufacturer of the IMD, a healthcare professional, or a patient and technical service provider with access to the IMD. For example, the external source can be a manufacturer call center located anywhere in the world. The patient or the healthcare professional can communicate or transmit the authentication code to the external source via telephone or another electronic device. Alternatively, the programmer application can automatically transmit the authentication code to the external source without any user intervention (e.g., the authentication code can be automatically generated and transmitted by the programmer application upon observing an unexpected operational state; alternatively, the user can request generation of the authentication code and the programmer application can automatically transmit the code to the external source).

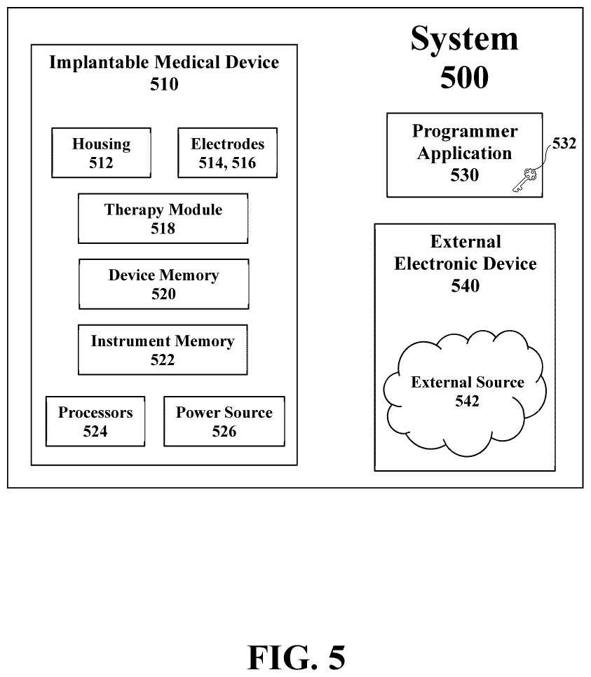

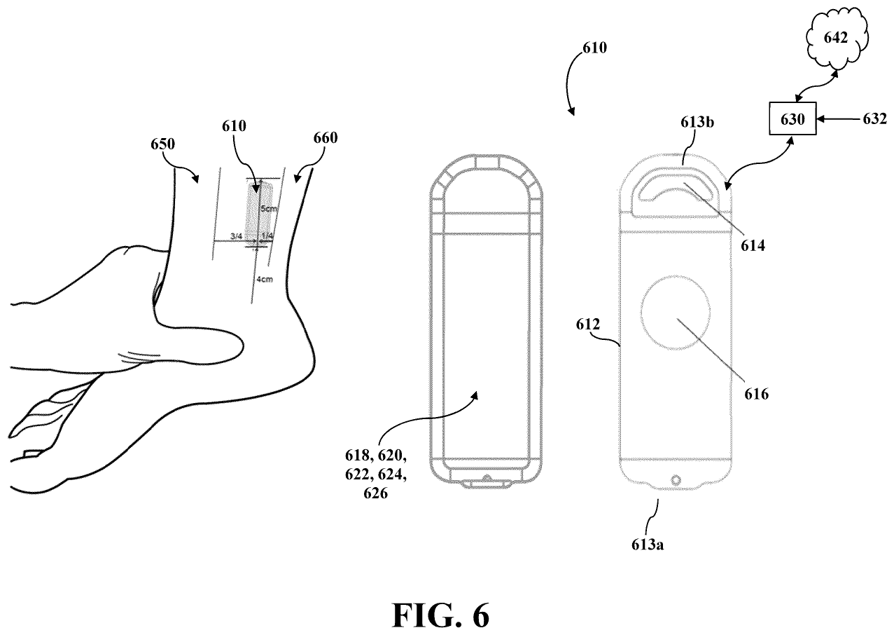

Upon receiving the authentication code, the external source can determine whether an IMD recovery operation is needed or whether standard, built-in troubleshooting or debugging measures can be used to resolve the reported issue. The external source can also log or record the expected or unexpected operational state before making the recovery determination. If the external source determines that the CPD feature is necessary, the external source can send an authentication key, corresponding to the transmitted authentication code, to the programmer application. The authentication key can be generated using a program such as Rosetta or another similar secure key generation program. The authentication key generator program and the programmer application can have a shared, secret process for generating an authentication key corresponding to the authentication code (e.g., the programmer application can contain a secret key, such as secret keys 532, 632 illustrated in FIGS. 5 and 6, respectively, to verify the authentication key generated by the external source, with the secret key corresponding to the authentication key).

At 106, the programmer application can receive the authentication key sent by the external source. The external source can send the authentication key to the programmer application over electronic communication, for example via electronic mail, notification through an electronic device, or via telephone communication between the external source and a user of the programmer application. The programmer application can be configured to receive the authentication key simultaneously, or close to simultaneously, as the key is generated by the external source. In examples, there can be a programmed delay between the external source generating the authentication key and the programmer application receiving the key (e.g., five minutes, one hour, one day). This can provide a further security layer for preventing an unintentional or unneeded CPD recovery operation by giving the user or the external source additional time to attempt other correction mechanisms.

At 108, the programmer application can read the authentication key sent by the external source to validate the transmitted authentication code. Reading the authentication key can comprise comparing the authentication key received from the external source to a secret key stored within the programmer application. The secret key is generally found in source code of the programmer application, in a secure location secured by encryption technology, or in a properties file located in assets of the programmer application. The secret key may be found in other encrypted locations of the programmer application in place of or in addition to the source code and properties file locations.

If the received authentication key matches the secret key, the programmer application can validate the authentication code and the IMD recovery operation can proceed (e.g., device reset and reprogramming while retaining existing service life and existing diagnostic data information). If the received authentication key does not match the secret key, the programmer application may reread or reenter the received key until a match is found. The programmer application can automatically reread or reenter the key until a match is found, or the user can perform this step manually. In examples, the programmer application can be configured to generate a retry limit exceeded indication if the received key is entered five times without matching the copied key. In such cases, the programmer application can be further configured to generate a new authentication code upon receiving the retry limited exceeded indication, and to transmit the new authentication code to the external source configured to generate a new authentication key. The authentication key reading and, if necessary, rereading processes described previously can then be repeated for the new key. In examples, the number of attempts before a new authentication code is generated may be greater or less than five attempts, depending on what value is programmed into the programmer application (e.g., the programmer application may be programmed to allow three attempts or ten attempts before a new authentication code is generated).

At 110, upon validating the authentication code, the programmer application can reset one or more device operation settings stored in a device memory of the IMD to a default parameter. Existing service life and existing diagnostic data of the IMD are saved in the device memory rather than being reset. For example, the programmer application can be configured to read existing service life information from a device memory of the IMD prior to implementing the CPD recovery operation. In examples, the one or more device operation settings may comprise therapy scheduler settings, stimulation settings, instrument memory information stored within the IMD, patient information, a magnetic resonance imaging (MRI) mode that enables the IMD to safely operate during an MRI scan, and other similar IMD or programmer application settings or information. Device operation settings may vary depending on the type of IMD being used (e.g., a neurostimulator may have different device operations settings and parameters compared to an implantable cardioverter defibrillator). In examples, the device operation settings can be reset or cleared regardless of whether they are operating in expected or unexpected states. This ensures that all unexpected device operations settings are corrected during a recovery operation rather than only the observed unexpected or invalid state(s) or setting(s). In examples, existing diagnostic data can be read from the device memory using the programmer application.

At 112, the existing service life saved in the device memory can be written to an instrument memory of the IMD. In particular, after resetting or clearing all or some device operation settings, the programmer application can be configured to write default or existing therapy parameters for these settings and configured to write the saved existing service life information, all into an instrument memory of the IMD. The instrument memory is generally cleared or reset prior to receiving the service life information. In examples, the existing diagnostic data can be written to the instrument memory along with the existing service life information. Generally, the instrument memory differs from the device memory in that when the instrument memory experiences an invalid data field or a corrupted outcome, existing service life and existing diagnostic data information in the device memory are still maintained. This enables the existing service life information stored in the device memory to be written to the instrument memory to return the IMD to an expected operational state.

Accordingly, first method 100 enables recovery of an IMD from an unexpected operational state to an expected operational state while retaining existing service life and existing diagnostic data information which remain unchanged during the recovery operation. Recovery occurs by validating certain memory blocks in the device and instrument memories and resetting such blocks to default or existing values. Ideally, first method 100 is not used because the IMD is designed to operate within expected states without deviation. However, in the rare circumstance where an unexpected operational state is observed, first method 100 provides a correction process that is less costly and more convenient than conventional solutions such as IMD explant or field corrective actions which both require in-person intervention by a device manufacturer or a healthcare professional.

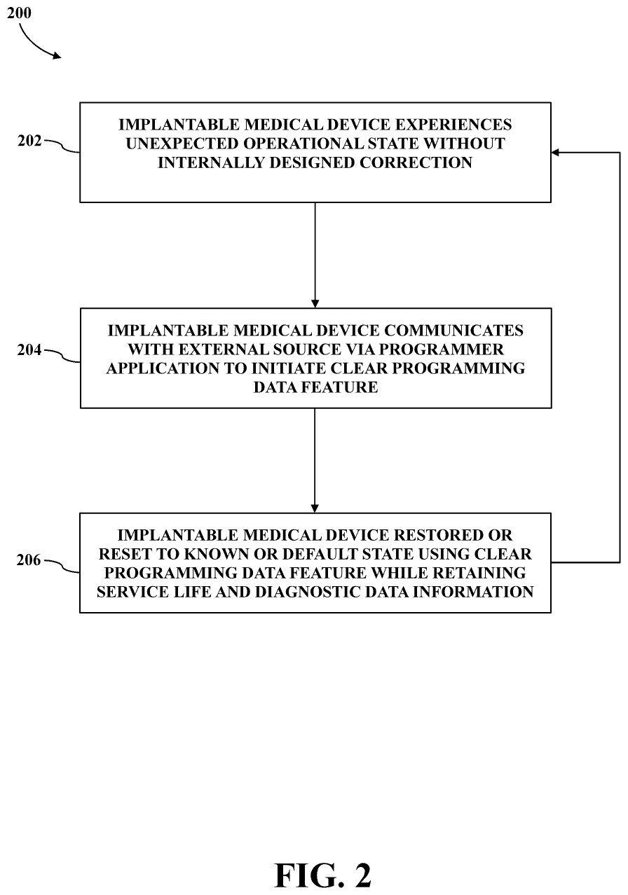

FIG. 2 illustrates a second method 200 for recovering an implantable medical device (IMD) from unexpected operational states, according to examples of the present disclosure. Second method 200 may be performed using a programmer application configured to restore the IMD to an expected operational state. The programmer application may be couplable (e.g., electrically, communicatively, integrally, etc.) to the IMD. The programmer application may comprise a built-in “clear programming data” (CPD) feature configured to reset one or more IMD operation settings (e.g., all settings, except for existing service life and existing diagnostic data, which undergo periodic validation checks or cyclic redundancy checks). The programmer application may be further configured to reprogram the IMD with default parameters corresponding to the one or more reset device operation settings, along with reprogramming the retained existing service life information into the IMD.

At 202, the IMD can experience a rare, unexpected operational state that does not have an internally designed correction. Examples of rare, unexpected operational states include invalid data fields not previously observed and corrupted device operation settings without a correction mechanism built-in to the programmer application. The unexpected operational state may be observed and recorded by the programmer application automatically, or may be observed by the user of the IMD and manually reported to the programmer application.

At 204, upon experiencing a rare, unexpected operational state, the IMD can communicate with an external source via the programmer application to initiate a clear programming data (CPD) feature to restore or reset the IMD to an expected operational state. If the unexpected operational state is observed automatically, the programmer application can be configured to automatically communicate with the external source to begin the process of initiating the CPD feature. If the unexpected operational state is observed by a user of the IMD, the user may manually request intervention by the external source using the programmer application or through an alternative electronic mode (e.g., electronic mail, electronic communication with the external source via a telephone, etc.).

At 206, the IMD can be restored or reset to an expected, known, or default operational state using the CPD feature while retaining existing service life and existing diagnostic data information. The restoration operation can be initiated and performed using the approach described for first method 100, with or without additional steps automatically performed by the programmer application or manually performed by the user. For example, second method 200 may include the process of first method 100 along with the additional steps of resetting the instrument memory prior to writing the existing service life to the instrument memory; reading the existing service life from the device memory prior to writing the existing service life to an instrument memory; and reading existing device information of the IMD prior to generating the authentication code. It should be understood that first method 100 can perform the same additional steps either individually or simultaneously with other steps described for first method 100.

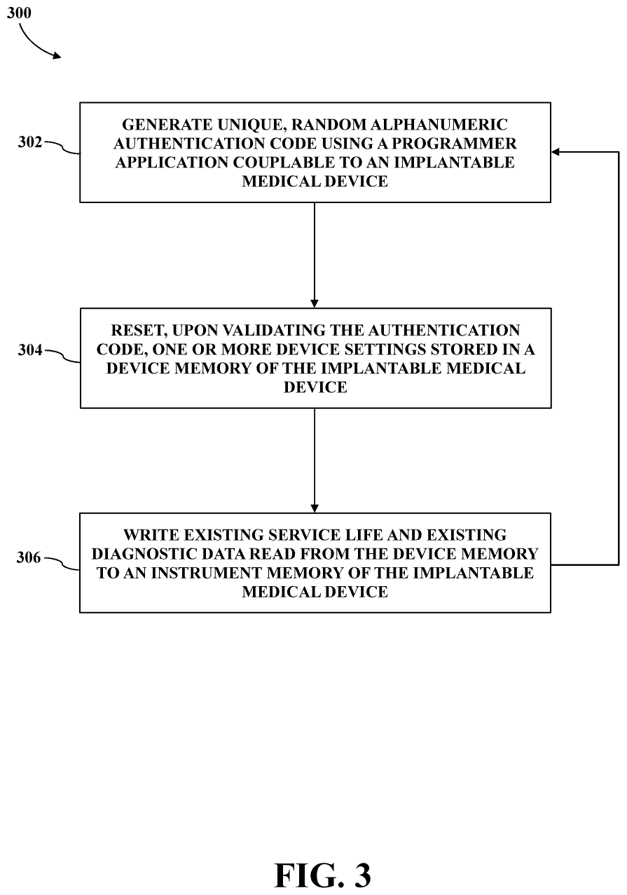

FIG. 3 illustrates a third method 300 for recovering an implantable medical device (IMD) from unexpected operational states, according to examples of the present disclosure. Third method 300 may be performed using a programmer application configured to restore the IMD to an expected operational state. The programmer application may be couplable (e.g., electrically, communicatively, integrally, etc.) to the IMD. The programmer application may comprise a built-in “clear programming data” (CPD) feature configured to reset one or more IMD operation settings (e.g., all settings, except for existing service life and existing diagnostic data, which undergo periodic validation checks or cyclic redundancy checks). The programmer application may be further configured to reprogram the IMD with default parameters corresponding to the one or more reset device operation settings, along with reprograming the retained existing service life into the IMD.

At 302, the programmer application can be configured to generate a unique, random alphanumeric authentication code corresponding to the IMD. As described previously, the authentication code can be a unique, randomly generated alphanumeric code comprising more than, less than, or exactly six characters. The authentication code can be stored in an encrypted location within the programmer application and not maintained beyond its initial storage in the encrypted location for a particular recovery operation. The authentication code can be randomly generated by a secure random character generator. The authentication code can be tied to an authentication key generated by the external source as described in 304. The authentication code can be validated using the generated authentication key.

At 304, upon validating the authentication code using an authentication key received from an external source, the programmer application can be configured to reset or clear one or more device operation settings stored in a device memory of the IMD. Existing service life and existing diagnostic data of the IMD are retained during this operation. The device operation settings can be reset or cleared regardless of whether they are operating in expected or unexpected states, to ensure that device recovery corrects all unexpected operational states, settings, or data fields regardless of whether they are known.

At 306, the programmer application can be configured to write the existing service life read from the device memory to an instrument memory of the IMD. The programmer application can also be configured to reprogram the cleared device operation settings to default or existing therapy parameters. This gives the IMD the appearance of being factory reset but with existing service life and existing diagnostic data information retained. Accordingly, third method 300 resets the IMD from an unexpected operational state to an expected operational state with core service life and diagnostic information left intact, all without requiring IMD explant or field corrective actions. As such, the IMD can continue operation after being reset while retaining the service life start date, expected date(s) of when device service is required, and various diagnostic data recorded during past therapy sessions with the patient.

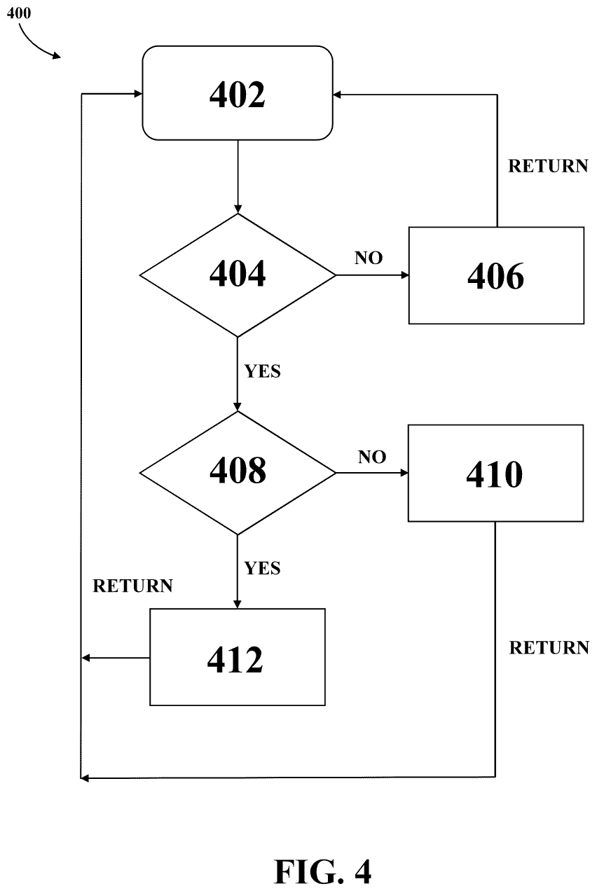

FIG. 4 illustrates a first flowchart 400 for recovering an implantable medical device (IMD) from unexpected operational states, according to examples of the present disclosure.

At 402, the IMD can begin normal operation in an expected operational state. At 404, a programmer application couplable to the IMD can determine whether the IMD has transitioned to an unexpected operational state. If the IMD is still operating in an expected operational state, then at 406 the IMD can continue normal operation and return to 402. However, if the IMD has transitioned to an unexpected operational state, then at 408 the programmer application can determine whether a standard built-in correction mechanism can remedy the issue to return the IMD to an expected operational state. If a standard built-in correction mechanism is unavailable, then at 410 the programmer application can initiate a recovery operation to reset or clear one or more device operation settings while retaining existing service life and diagnostic data information. After implementing the recovery operation, the IMD can continue normal operation and return to 402. If a standard built-in correction mechanism is available without requiring a recovery operation, then at 412 the programmer application can implement the standard correction mechanism to return the IMD to an expected operational state. After implementing the standard correction mechanism, the IMD can continue normal operation and return to 402.

FIG. 5 illustrates a block diagram of a system 500 configured to recover an implantable medical device (IMD) from unexpected operational states, according to examples of the present disclosure.

System 500 can comprise an IMD 510, a programmer application 530, and an external electronic device 540 having an external source 542 configured to communicate with the programmer application 530. IMD 510 can comprise a housing 512 extending between a proximal end and a distal end, a pair of electrodes 514, 516 (e.g., a stimulation electrode 514 and a return electrode 516) couplable to the housing 512, a therapy module 518 configured to deliver therapy to a patient (e.g., neurostimulation therapy), a device memory 520, an instrument memory 522, one or more processors 524, and a power source 526. Therapy module 518, device memory 520, instrument memory 522, one or more processors 524, and power source 526 may be received within a hollow region of housing 512. Alternatively, one or more of these components may be external to housing 512 in examples (e.g., external power source 526 configured to transmit power to the components of IMD 510 via inductive charging).

In examples, IMD 510 can comprise the programmer application 530 rather than programmer application being separate from IMD 510 (e.g., programmer application may be integrated into IMD 510). In either case, programmer application 530 can be configured to perform a recovery operation or a “clear programming data” (CPD) feature as described with respect to first, second, and third methods 100, 200, 300. Programmer application 530 can be configured to interface between device memory 520 and instrument memory 522. In particular, programmer application 530 can be configured to read device operation settings and existing service life information from device memory 520 and write this information to instrument memory 522 during a recovery operation. In other examples, programmer application 530 can be configured to read existing diagnostic data from device memory 520 and write this data to instrument memory 522 during a recovery operation. Programmer application 530 can comprise a secret key 532 that can be compared to an authentication key received from an external source 542 to verify the authentication key.

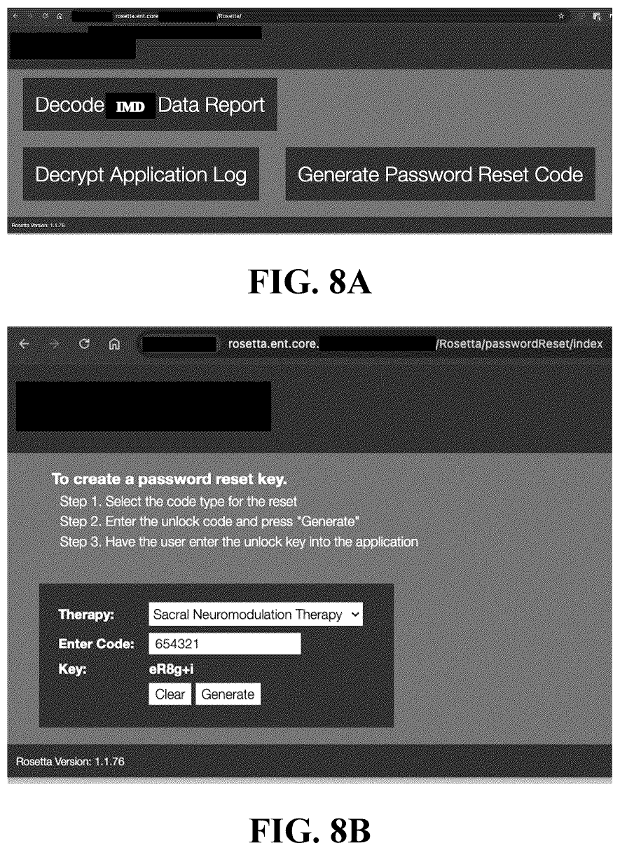

The programmer application 530 can be stored in a programming device, for example an electronic device such as a telephone or handset, a computer, a tablet, or a similar device configured to communicate with an IMD 510. In particular examples such as those illustrated in FIG. 8, a recharger communicator can be used as a communication relay between the programming device with programmer application 530 and the IMD 510 (e.g., communication between the application 530 and IMD 510 can occur via the recharger communicator). The recharger communicator is generally a wireless device external to the IMD 510. In other examples, the programming device with programmer application 530 can be configured to communicate directly with the IMD 510 without using the recharger communicator as a communication relay. For example, the IMD 510 can be couplable to the programmer application 530 via Bluetooth or a mobile device configured to send signals using a remote application on the mobile device.

The pair of electrodes 514, 516 can be configured to provide stimulation to a patient for therapeutic, pain management, or bodily recovery procedures. Therapy module 518 can be couplable to the pair of electrodes 514, 516 and configured to provide one or more therapy settings used by the pair of electrodes 514, 516. Device memory 520 can be configured to store IMD 510 operation settings, existing IMD 510 service life information, and existing IMD 510 diagnostic data. Instrument memory 522 can be configured to store the same IMD 510 information. The one or more processors 524 can be configured to control operation of IMD 510 and its associated components (e.g., pair of electrodes 514, 516 and therapy module 518). Power source 526 can be configured to supply power to IMD 510 to sustain device operation over time. In examples, power source 526 can comprise an internal or external battery that optionally can be recharged for continued use over time.

Device memory 520 can be configured to store operation settings including therapy scheduler settings, stimulation settings, instrument memory information stored within the IMD, patient information, a magnetic resonance imaging (MRI) mode that enables the IMD to safely operate during an MRI scan, and other similar IMD or programmer application settings or information. Device operation settings stored in device memory 520 may vary depending on the type of IMD being used (e.g., a neurostimulator may have different device operations settings and parameters compared to an implantable cardioverter defibrillator). Device memory 520 may also be configured to store existing service life and diagnostic data information.

Using the programmer application 530, device memory 520 can have one or more device operation settings reset during a recovery operation from an unexpected operational state. Existing service life and existing diagnostic data of the IMD can be retained in the device memory 520 rather than being reset with the device operation settings. The programmer application 530 can be configured to read existing service life from the device memory 520 prior to implementing the recovery operation. Subsequently, the existing service life saved in the device memory 520 can be written to the instrument memory 522. Moreover, after resetting or clearing all or some device operation settings in device memory 520, the programmer application can be configured to write default or existing therapy parameters for these settings, and write existing service life into the instrument memory 522. The instrument memory 522 is generally cleared or reset prior to receiving the service life information from the device memory 520. Generally, diagnostic data on the device memory 520 is read-only and thus is not reset or cleared during the recovery operation.

Instrument memory 522 differs from device memory 520 because existing service life and existing diagnostic data information are maintained in the device memory 520 even when instrument memory 522 experiences an invalid data field or a corrupted outcome because of an unexpected operational state. This enables the existing service life information saved in the device memory 520 to be written to the instrument memory 522 to return the IMD 510 to an expected operational state during a recovery operation. Accordingly, the recovery operation or CPD feature validates certain memory blocks in device memory 520 and instrument memory 522 while resetting or clearing such memory blocks to default or existing values. The existing service life and existing diagnostic data information, which are both stored in the device memory 520, remain unchanged during the recovery operation. Furthermore, in some examples, certain existing service life information from device memory 520 is redundantly written or copied into instrument memory 522 to address circumstances not directly related to the recovery operation.

In other examples, the programmer application 530 can be configured to read existing diagnostic data information from the device memory 520 prior to implementing the recovery operation. In other examples, the existing diagnostic data saved in the device memory 520 can be written to the instrument memory 522 during the recovery operation.

The specific features, functionality, and designs of IMD 510 are not the focus of the present disclosure. However, it should be understood that IMD 510 can comprise a variety of implantable devices including a neurostimulator, an implantable cardiac defibrillator, hearing aid implants, joint implants, brain implants, spinal stimulators, sacral neuromodulation devices, deep brain stimulators, obstructive sleep apnea neurostimulators, and pacemakers, for example. Other implantable devices configured to provide therapy, pain management, or bodily recovery to a patient that are known to one of ordinary skill in the art may be used with IMD 510.

FIG. 6 illustrates an implantable medical device (IMD) 610 configured to recover from unexpected operational states, according to examples of the present disclosure.

IMD 610 has many equivalent and similar features compared to IMD 510 and for simplicity the description of common components is not repeated in the following. Like reference numerals and features names may designate like feature names throughout that are corresponding or analogous.

In examples where IMD 610 is a neurostimulator, IMD 610 can be implanted into a patient according to the following process. The implant location can involve an IMD 610 with a center point between approximately four and six centimeters superior to a midpoint of the patient medial malleolus along a length of IMD 610 parallel to the patient tibia and the Achilles tendon. Generally, a centerline of IMD 610 should be approximately 75% of a distance between a posterior border of the patient tibia and 25% of a distance toward an anterior border of the patient Achilles tendon. A healthcare professional or clinician can position IMD 610 so that a stimulation electrode 614 at a distal end 613b of the housing 612 is facing toward a fascia and nerve of the patient, and an active electrode 616 at a proximal end 613a of the housing 612 is pointed toward a patient foot. In such examples, tibial neurostimulation of the patient can be performed to remedy pelvic health maladies after implanting IMD 610.

IMD 610 can further comprise a programmer application 630 configured to perform a recovery operation or a “clear programming data” (CPD) feature as described with respect to first, second, and third methods 100, 200, 300. In examples, the programmer application 630 can be stored on a programming device such as a handset or another device configured for communication with a wireless recharger communicator or the IMD 610. Programmer application 630 can comprise a secret key 632 that can be compared to an authentication key received from an external source 642 to verify the authentication key. In examples, the programmer application 630 can communicate directly with the IMD 610 via Bluetooth or mobile device signals (e.g., signals delivered using a mobile device remote application outside the Bluetooth range). Programmer application 630 can be configured to interface between a device memory 620 and an instrument memory 622 of IMD 610. In particular, programmer application 630 can be configured to read device operation settings, existing service life information, and optionally existing diagnostic data from device memory 620 and write this information to instrument memory 622 during a recovery operation from an unexpected operational state. Further discussion of recovery operations using the CPD recovery feature is provided in reference to methods 100, 200, 300 discussed previously.

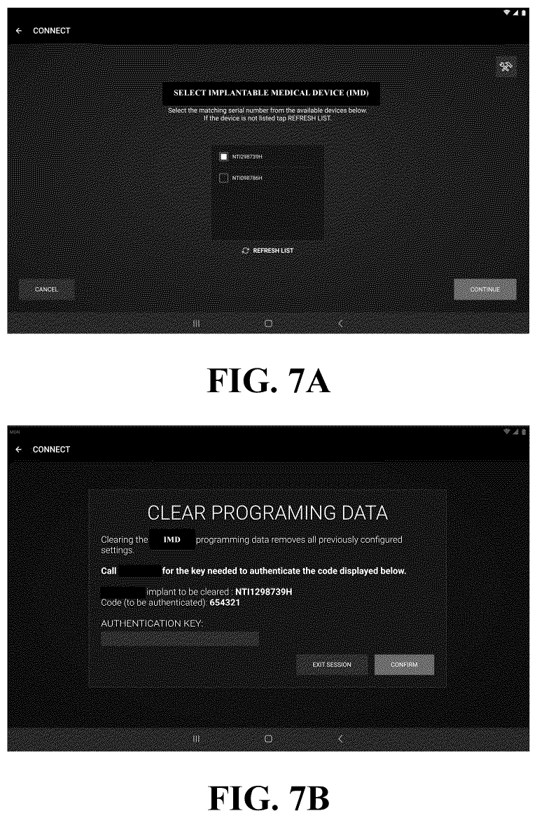

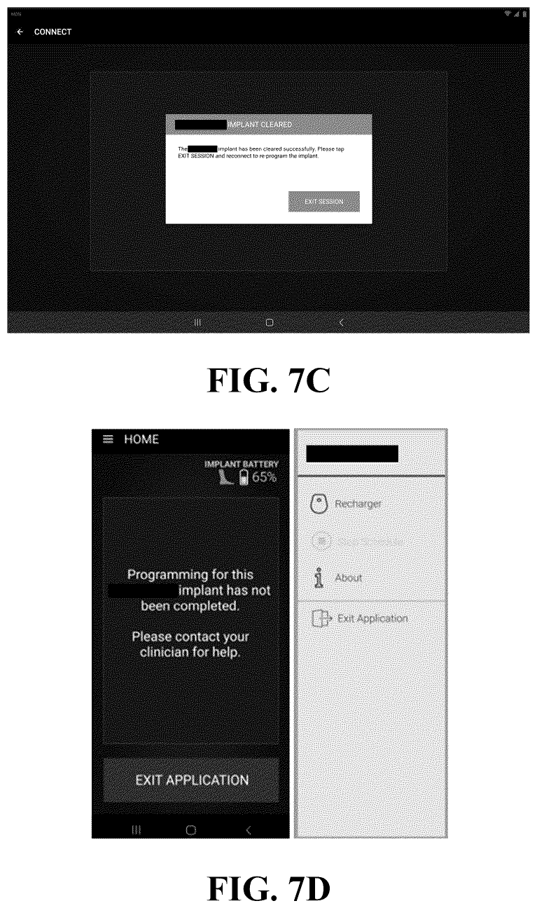

FIGS. 7A-D illustrate graphical user interface (GUI) screens of a programmer application configured for use with an implantable medical device (IMD), according to examples of the present disclosure.

Referring to FIG. 7A, the programmer application can prompt a user such as a patient or a healthcare professional to select a serial number or other indicator corresponding to a unique IMD. This selection can occur prior to starting an operation to recover an IMD from an unexpected operational state or from another device challenge. The programmer application can be couplable to one or more unique IMDs specific to the user, with the unique IMDs listed by serial number in a selection box included with the programmer application. As illustrated in FIG. 7A, a user can choose between a first IMD with serial number “NTI298739H” and a second IMD with serial number “NTI098786H.” In examples, the first IMD can be a neurostimulator while the second IMD can be an implantable cardioverter defibrillator. In other examples, the first and second IMDs can both be neurostimulators implanted in different bodily locations of a patient.

To start a recovery operation, a user can first select the IMD to be recovered from the selection box, and then press a “CONTINUE” button to advance the GUI to a subsequent screen in the programmer application illustrated in FIG. 7B. In examples, the programmer application will first determine that the IMD has sufficient battery power and accurate telemetry security before advancing to the subsequent screen. If the user determines that IMD recovery is no longer needed, the user can press a “CANCEL” button which returns the programmer application to a home screen where other device operations can be performed. An IMD that was recently coupled to the programmer application can be added to the selection box by pressing a “REFRESH LIST” button which updates the listing of IMDs available for recovery operations.

Referring to FIG. 7B, the subsequent screen after pressing the “CONTINUE” button may provide an authentication code and a text box for entering an authentication key. The authentication code may be generated by an application programming interface (API) such as Harmony API. The authentication code and the authentication key may be generated or received as previously discussed with respect to other examples of the present disclosure. The authentication key can be received from an external source and entered into an “AUTHENTICATION KEY” box as illustrated. Once the authentication key is entered into the text box, a user can press a “CONFIRM” button which instructs the processor application to compare the entered authentication key to a secret key stored in an encrypted location within the programmer application. If the entered authentication key matches the secret key, the programmer application performs the recovery operation (e.g., “CLEAR PROGRAMMING DATA”) and then advances the GUI to a subsequent screen illustrated in FIG. 7C. Similar to FIG. 7A, if the user determines that IMD recovery is no longer needed, the user can press a first “EXIT SESSION” button which returns the programmer application to the home screen. A subsequent IMD recovery operation can be selected from the home screen if needed after exiting the previous session.

Referring to FIG. 7C, the subsequent screen after pressing the “CONFIRM” button may provide a confirmation message box that the IMD recovery operation was successful (e.g., “IMPLANT CLEARED”). The confirmation message box may include a second “EXIT SESSION” button that a user can press to return the programmer application to the home screen. Pressing the second “EXIT SESSION” button can also instruct the programmer application to reconnect to the IMD if the connection was lost, and to reprogram or reset one or more device operation settings of the IMD. The one or more device operation settings can be reprogramed or reset as previously discussed with respect to other examples of the present disclosure (e.g., reset to default or existing therapy parameters).

Referring to FIG. 7D, the home screen of the programmer application can provide a status box indicating whether IMD reprogramming is finished or still in progress after completing the recovery operation. The status box may also be used to indicate to a user that IMD recovery operations are not available because the IMD has not been initially programmed or has not been used for a given time period (e.g., 24 hours). Once the IMD is used for the given time period, which may be programmed into the programmer application, the status box may instruct the user that IMD recovery operations are now available. The user can then proceed to start the recovery operations as discussed with respect to FIGS. 7A-C and with respect to previous examples of the present disclosure.

FIGS. 8A and 8B illustrate GUI screens of an authentication key generator program for use with a programmer application and an implantable medical device, according to examples of the present disclosure. In particular examples, the authentication key generator program can comprise the Rosetta program. In particular examples, the authentication key generator program can be accessible by an external source that can generate an authentication key upon determining that IMD recovery using the CPD feature is necessary.

Referring to FIG. 8A, the authentication key generator program can be configured to decode a data report associated with an IMD by selecting a “Decode IMD Data Report” button. The authentication key generator program can be further configured to decrypt an application log associated with the program by selecting a “Decrypt Application Log” button. Most notably for the present disclosure, the authentication key generator program can be further configured to generate an authentication key based on an authentication code generated and transmitted by the programmer application. A GUI for generating the authentication key can be accessed by selecting a “Generate Password Reset Code” button. Selecting this button transitions the authentication key generator program to the GUI illustrated in FIG. 8B.

Referring to FIG. 8B, the authentication key generator program can provide instructions for generating an authentication key based on an authentication code transmitted by the programmer application. The instructions can comprise “Step 1. Select the code type for the reset,” “Step 2. Enter the unlock code and press ‘Generate,’” and “Step 3. Have the user enter the unlock key into the application.” Following the instructions, the authentication key generator program can include a therapy dropdown box to select a therapy type corresponding to the IMD (e.g., sacral neuromodulation therapy) and a code box for entering the authentication code transmitted by the programmer application. Selecting the “Generate” button enables the authentication key generator program to generate the authentication key corresponding to the transmitted authentication code. The generated authentication key can be entered in the “AUTHENTICATION KEY” box illustrated in FIG. 7B to activate the CPD recovery feature. The therapy type, the entered authentication code, and the generated authentication key can be reset or erased using the “Clear” button positioned proximate the “Generate” button. This is useful for when the therapy type or authentication code were entered incorrectly, or when a new therapy type or authentication code is necessary.

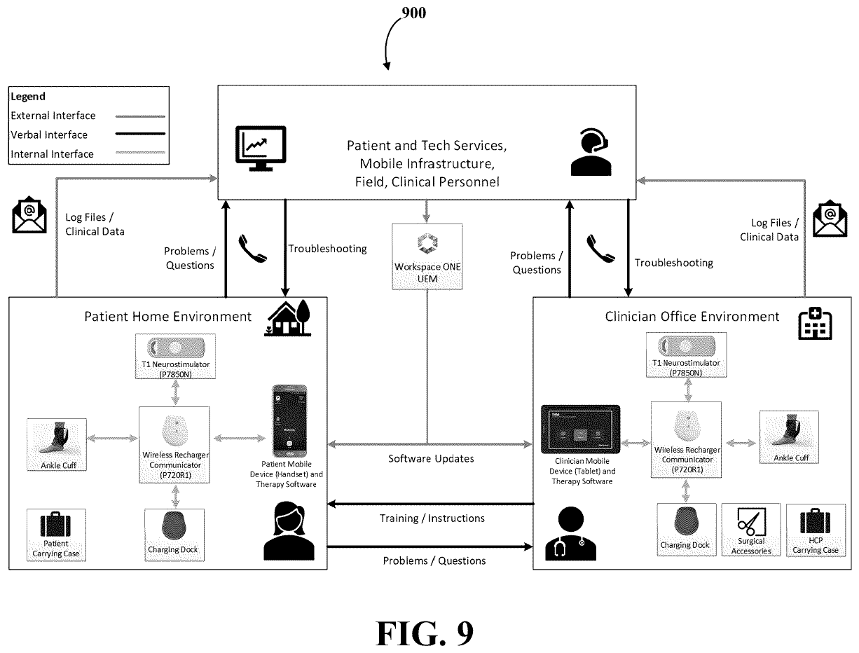

FIG. 9 illustrates a second flowchart 900 for recovering an implantable medical device (IMD) from unexpected operational states.

Second flowchart 900 depicts external, verbal, and internal interfaces between patient and technical services, mobile infrastructure, and field and clinical personnel (hereafter “technical services”); a patient home environment; and a clinician office environment. The interfaces, relationships, systems, and devices depicted in second flowchart 900 may be used in conjunction with methods for recovering an implantable medical device from unexpected operational states as described herein. Specifically, second flowchart 900 depicts example interactions between a patient, a clinician, and an IMD manufacturer that may occur during a recovery operation.

The patient home environment can comprise an IMD such as a neurostimulator, an ankle cuff to be worn by the patient, a wireless recharger communicator, a charging dock, a patient carrying storage case, and a patient mobile or electronic device with therapy software. The therapy software may comprise a programmer application as described previously.

The clinician office environment can comprise an IMD such as a neurostimulator, an ankle cuff to be worn by the patient, a wireless recharger communicator, a charging dock, a carrying and storage case, various surgical accessories, and a clinician mobile or electronic device with therapy software. As with the patient device, the therapy software of the clinician device may comprise a programmer application as described previously.

The patient home environment can be configured to transmit IMD log files and clinical data to technical services. The patient home environment can be configured to transmit problems or questions to technical services. Technical services can be configured to provide troubleshooting to resolve any problems or questions transmitted by the patient home environment. Technical services can perform the same operations with the clinician office environment. Technical services can provide software updates to the patient and clinician devices, for example to provide permanent solutions to unexpected operational states temporarily resolved with a recovery operation. The patient home environment can be configured to transmit problems or questions to the clinician office environment. The clinician office environment can be configured to provide training or instructions to resolve any problems or questions transmitted by the patient home environment.

According to a first example of the present disclosure, a method for recovering an implantable medical device (IMD) from unexpected operational states is provided. The method can include generating, from a programmer application, an authentication code corresponding to the IMD; transmitting, via the programmer application, the authentication code to an external source; receiving, via the programmer application, an authentication key from the external source; reading, via the programmer application, the authentication key to validate the authentication code; resetting, upon validating the authentication code, one or more device operation settings stored in a device memory of the IMD, wherein existing service life and existing diagnostic data of the IMD are maintained in the device memory during the reset; and writing, from the device memory, the existing service life and at least one default parameter corresponding to the one or more reset device operation settings to an instrument memory of the IMD.

In a second example of the method of the first example or any other example, the method can further include resetting the instrument memory prior to writing the existing service life and the at least one default parameter corresponding to the one or more device operation settings to the instrument memory.

In a third example of the method of the first example or any other example, the method can further include reading the existing service life from the device memory prior to writing the existing service life to the instrument memory.

In a fourth example of the method of the first example or any other example, the one or more device operation settings can include therapy scheduler settings, stimulation settings, patient information, and a magnetic resonance imaging (MRI) mode that enables the IMD to safely operate during an MRI scan.

In a fifth example of the method of the first example or any other example, the authentication code can be a unique, random alphanumeric code that is not maintained by the programmer application.

In a sixth example of the method of the first example or any other example, a secret key can be stored in an encrypted location within the programmer application, wherein validating the authentication code can include comparing the authentication key received from the external source to the secret key stored within the programmer application.

In a seventh example of the method of the first example or any other example, the external source can be a manufacturer of the IMD or a healthcare provider.

In an eighth example of the method of the first example or any other example, the method can further include reading, via the programmer application, existing device information of the IMD prior to generating the authentication code, wherein the programmer application can be configured to generate the authentication code upon observing an unexpected operational state.

In a ninth example of the method of the first example or any other example, the programmer application can be configured to perform data validation checks of the IMD to ensure the IMD is operating in an expected operational state.

In a tenth example of the method of the first example or any other example, the method can further include repeating reading the authentication key if the authentication code is not validated, wherein the programmer application can be configured to generate a retry limit exceeded indication upon the authentication code not being validated after reading the authentication key a set number of times, wherein the programmer application can be configured to generate a new authentication code upon receiving the retry limited exceeded indication.

In an eleventh example of the method of the first example or any other example, the IMD can be a neurostimulator.

According to a twelfth example of the present disclosure, a system is provided. The system can include a programmer application and an implantable medical device (IMD). The IMD can include a device memory and an instrument memory. The programmer application can be configured to generate an authentication code corresponding to the IMD; transmit the authentication code to an external source; receive an authentication key from the external source; read the authentication key to validate the authentication code; reset, upon validating the authentication code, one or more device operation settings stored in the device memory, wherein existing service life and existing diagnostic data of the IMD are maintained in the device memory during the reset; and write, from the device memory, the existing service life and at least one default parameter corresponding to the one or more reset device operation settings to the instrument memory.

In a thirteenth example of the method of the twelfth example or any other example, the programmer application can be further configured to reset the instrument memory prior to writing the existing service life and the at least one default parameter corresponding to the one or more reset device operation settings to the instrument memory.

In a fourteenth example of the method of the twelfth example or any other example, the programmer application can be further configured to read the existing service life from the device memory prior to writing the existing service life to the instrument memory.

In a fifteenth example of the method of the twelfth example or any other example, the authentication code can be a unique, random alphanumeric code that is not maintained by the programmer application.

In a sixteenth example of the method of the twelfth example or any other example, a secret key can be stored in an encrypted location within the programmer application, wherein validating the authentication code can include comparing the authentication key received from the external source to the secret key stored within the programmer application.

According to a seventeenth example of the present disclosure, an implantable medical device (IMD) is provided. The IMD can include including a device memory having one or more device operation settings and an instrument memory. The device memory can be configured to reset the one or more device operation settings upon being instructed to. Existing service life and existing diagnostic data of the IMD can be maintained in the device memory during the reset. The instrument memory can be configured to receive the existing service life and at least one default parameter corresponding to the one or more reset device operation settings from the device memory.

In an eighteenth example of the IMD of the seventeenth example or any other example, the instrument memory can be configured to be reset prior to receiving the existing service life and the at least one default parameter corresponding to the one or more reset device operation settings from the device memory.

In a nineteenth example of the IMD of the seventeenth example or any other example, the device memory can be configured to reset the one or more device operation settings upon being instructed to by a programmer application configured to implement a recovery operation to recover the IMD from an unexpected operational state.

In a twentieth example of the IMD of the seventeenth example or any other example, resetting the device memory can be initiated after validating an authentication key sent by an external source, wherein the authentication key can be generated based on an authentication code corresponding to the IMD.

According to a twenty-first example of the present disclosure, a non-transitory computer-readable medium storing a set of instructions executable by a processor is provided. The set of instructions, when executed by the processor, cause the processor to: generate an authentication code corresponding to an implantable medical device (IMD); transmit the authentication code to an external source; receive an authentication key from the external source; read the authentication key to validate the authentication code; reset, upon validating the authentication code, one or more device operation settings stored in a device memory of the IMD, wherein existing service life and existing diagnostic data of the IMD are maintained in the device memory during the reset; and write, from the device memory, the existing service life and at least one default parameter corresponding to the one or more reset device operation settings to an instrument memory of the IMD.

In a twenty-second example of the non-transitory computer-readable medium of the twenty-first example or any other example, the set of instructions, when executed by the processor, further cause the processor to reset the instrument memory prior to writing the existing service life and the at least one default parameter corresponding to the one or more device operation settings to the instrument memory.

In a twenty-third example of the non-transitory computer-readable medium of the twenty-first example or any other example, the set of instructions, when executed by the processor, further cause the processor to read the existing service life from the device memory prior to writing the existing service life to the instrument memory.

In a twenty-fourth example of the non-transitory computer-readable medium of the twenty-first example or any other example, the one or more device operation settings comprise therapy scheduler settings, stimulation settings, patient information, and a magnetic resonance imaging (MRI) mode that enables the IMD to safely operate during an MRI scan.

In a twenty-fifth example of the non-transitory computer-readable medium of the twenty-first example or any other example, the authentication code is a unique, random alphanumeric code that is not maintained by the processor.

In a twenty-sixth example of the non-transitory computer-readable medium of the twenty-first example or any other example, a secret key is stored in an encrypted location within the processor, wherein validating the authentication code comprises comparing the authentication key received from the external source to the secret key stored within the processor.

In a twenty-seventh example of the non-transitory computer-readable medium of the twenty-first example or any other example, the external source is a manufacturer of the IMD or a healthcare provider.

In a twenty-eighth example of the non-transitory computer-readable medium of the twenty-first example or any other example, the set of instructions, when executed by the processor, further cause the processor to: read existing device information of the IMD prior to generating the authentication code; and generate the authentication code upon observing an unexpected operational state of the IMD.

In a twenty-ninth example of the non-transitory computer-readable medium of the twenty-first example or any other example, the set of instructions, when executed by the processor, further cause the processor to perform data validation checks of the IMD to ensure the IMD is operating in an expected operational state.

In a thirtieth example of the non-transitory computer-readable medium of the twenty-first example or any other example, the set of instructions, when executed by the processor, further cause the processor to: repeat reading the authentication key if the authentication code is not validated; generate a retry limit exceeded indication upon the authentication code not being validated after reading the authentication key a set number of times; and generate a new authentication code upon receiving the retry limited exceeded indication.

In a thirty-first example of the non-transitory computer-readable medium of the twenty-first example or any other example, the IMD is a neurostimulator.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

1. A method for recovering an implantable medical device (IMD) from unexpected operational states, comprising:
generating, from a programmer application, an authentication code corresponding to the IMD;
transmitting, via the programmer application, the authentication code to an external source;
receiving, via the programmer application, an authentication key from the external source;
reading, via the programmer application, the authentication key to validate the authentication code;
resetting, upon validating the authentication code, one or more device operation settings stored in a device memory of the IMD, wherein existing service life and existing diagnostic data of the IMD are maintained in the device memory during the reset; and
writing, from the device memory, the existing service life and at least one default parameter corresponding to the one or more reset device operation settings to an instrument memory of the IMD.

2. The method of claim 1, further comprising resetting the instrument memory prior to writing the existing service life and the at least one default parameter corresponding to the one or more device operation settings to the instrument memory.

3. The method of claim 1, further comprising reading the existing service life from the device memory prior to writing the existing service life to the instrument memory.

4. The method of claim 1, wherein the one or more device operation settings comprise therapy scheduler settings, stimulation settings, patient information, and a magnetic resonance imaging (MRI) mode that enables the IMD to safely operate during an MRI scan.

5. The method of claim 1, wherein the authentication code is a unique, random alphanumeric code that is not maintained by the programmer application.

6. The method of claim 1, wherein a secret key is stored in an encrypted location within the programmer application, wherein validating the authentication code comprises comparing the authentication key received from the external source to the secret key stored within the programmer application.

7. The method of claim 1, wherein the external source is a manufacturer of the IMD or a healthcare provider.

8. The method of claim 1, further comprising reading, via the programmer application, existing device information of the IMD prior to generating the authentication code, wherein the programmer application is configured to generate the authentication code upon observing an unexpected operational state of the IMD.

9. The method of claim 1, wherein the programmer application is configured to perform data validation checks of the IMD to ensure the IMD is operating in an expected operational state.

10. The method of claim 1, further comprising repeating reading the authentication key if the authentication code is not validated, wherein the programmer application is configured to generate a retry limit exceeded indication upon the authentication code not being validated after reading the authentication key a set number of times, wherein the programmer application is configured to generate a new authentication code upon receiving the retry limited exceeded indication.

11. The method of claim 1, wherein the IMD is a neurostimulator.

12. A system comprising:
a programmer application; and
an implantable medical device (IMD), wherein the IMD comprises:
a device memory; and
an instrument memory,
wherein the programmer application is configured to:
generate an authentication code corresponding to the IMD;
transmit the authentication code to an external source;
receive an authentication key from the external source;
read the authentication key to validate the authentication code;
reset, upon validating the authentication code, one or more device operation settings stored in the device memory, wherein existing service life and existing diagnostic data of the IMD are maintained in the device memory during the reset; and
write, from the device memory, the existing service life and at least one default parameter corresponding to the one or more reset device operation settings to the instrument memory.

13. The system of claim 12, wherein the programmer application is further configured to reset the instrument memory prior to writing the existing service life and the at least one default parameter corresponding to the one or more reset device operation settings to the instrument memory.

14. The system of claim 12, wherein the programmer application is further configured to read the existing service life from the device memory prior to writing the existing service life to the instrument memory.

15. The system of claim 12, wherein the authentication code is a unique, random alphanumeric code that is not maintained by the programmer application.

16. The system of claim 12, wherein a secret key is stored in an encrypted location within the programmer application, wherein validating the authentication code comprises comparing the authentication key received from the external source to the secret key stored within the programmer application.

17. An implantable medical device (IMD), comprising:
a device memory comprising one or more device operation settings; and
an instrument memory,
wherein the device memory is configured to reset the one or more device operation settings upon being instructed to, wherein existing service life and existing diagnostic data of the IMD are maintained in the device memory during the reset, and
wherein the instrument memory is configured to receive the existing service life and at least one default parameter corresponding to the one or more reset device operation settings from the device memory.

18. The IMD of claim 17, wherein the instrument memory is configured to be reset prior to receiving the existing service life and the at least one default parameter corresponding to the one or more reset device operation settings from the device memory.

19. The IMD of claim 17, wherein the device memory is configured to reset the one or more device operation settings upon being instructed to by a programmer application configured to implement a recovery operation to recover the IMD from an unexpected operational state.

20. The IMD of claim 17, wherein resetting the device memory is initiated after validating an authentication key sent by an external source, wherein the authentication key is generated based on an authentication code corresponding to the IMD.
